(19) United States
(12) Patent Application Publication
(10) Pub. No.: US 2026/0232940 A1
(43) Pub. Date: Aug. 13, 2026

(54) POSITIONING AND STABILISING STRUCTURE FOR A PATIENT INTERFACE

(71) Applicant: ResMed Asia Pte. Ltd., Singapore (SG)

(72) Inventors: Rahul KHERA, Sydney (AU); Sabine KONZACK, Sydney (AU); Yan Seng LIM, Singapore (SG); Skye Kimberley SHARMA, Sydney (AU); Thontira SUPAOPASPHUN, Singapore (SG); Alexandra Elizabeth TILSON, Sydney (AU)

(21) Appl. No.: 19/470,734

(22) PCT Filed: Apr. 1, 2024
(86) PCT No.: PCT/SG2024/050211

§ 371 Date: Sep. 29, 2025

(30) Foreign Application Priority Data
Mar. 31, 2023 (AU) 2023900902
Aug. 28, 2023 (AU) 2023902766

(51) Int. Cl. A61M 16/06

(52) U.S. Cl. CPC A61M 16/0683; A61M 16/0616; A61M 2205/3344

(57) ABSTRACT

A positioning and stabilising structure for a patient interface for configured to deliver breathable gas at a therapeutic pressure to an entrance of a patient's airways. The structure may comprise rear and two side portions. The side portions may comprise a rigidiser arm positioned inside a sleeve. The rigidiser arm and the sleeve may be connected together so that substantially no part of the sleeve can move relative to the rigidiser arm. Each of the first and second side portions may be tapered such that their posterior end is wider than the anterior end.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Discase (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, c.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 1.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in scaling relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 1.2.3.1.3 Pressurised Air Conduit

In one type of treatment system, a flow of pressurised air is provided to a patient interface through a conduit in an air circuit that fluidly connects to the patient interface so that, when the patient interface is positioned on the patient's face during use, the conduit extends out of the patient interface forwards away from the patient's face. This may sometimes be referred to as a “tube down” configuration.

Some patients find such interfaces to be unsightly or to create a feeling of claustrophobia and are consequently deterred from wearing them, reducing patient compliance. Additionally, conduits connecting to an interface at the front of a patient's face may sometimes be vulnerable to becoming tangled up in bed clothes.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

According to one aspect of the present technology, there is provided a positioning and stabilising structure for a patient interface configured to deliver breathable gas at a therapeutic pressure of at least 4 cmH₂O above ambient air pressure to an entrance to a patient's airways. The positioning and stabilising structure may comprise a rear portion configured to overlie a posterior region of the patient's head. The positioning and stabilising structure may further comprise first and second side portions, each being configured, in use, to extend across a respective cheek region of the patient's head, and each having a posterior end connected to a respective side of the rear portion and an anterior end configured to connect directly or indirectly to a seal-forming structure of the patient interface, the seal-forming structure being to form a seal with a region of the patient's face surrounding an entrance to the patient's airways. Each of the first and second side portions may comprise a rigidiser arm positioned inside a sleeve.

In certain forms, the rigidiser arm and the sleeve may be connected together so that substantially no part of the sleeve can move relative to the rigidiser arm.

In certain forms, the first and second side portions may be each tapered such that the posterior end is wider than the anterior end.

In certain forms, the sleeve is formed so as to be flexible.

In certain forms, the sleeve is formed from a textile material.

In certain forms, the sleeve may comprise an outer sleeve and an inner sleeve. The inner sleeve may be positioned inside the outer sleeve and the rigidiser arm may be positioned inside the inner sleeve.

In certain forms, the inner sleeve is thicker than the outer sleeve.

In certain forms, the inner sleeve is denser than the outer sleeve.

In certain forms, the inner sleeve is formed with a denser weave or knit pattern than the outer sleeve.

In certain forms, each of the first and second side portions may comprise one or more cushion portions positioned inside the sleeve. For example, each of the first and second side portions may comprise two cushion portions positioned inside the sleeve on opposite lateral sides of the rigidiser arm.

In certain forms, the rigidiser arm may be formed from a heat-activated material. The heat-activated material may be bonded with the sleeve.

In certain forms, the rigidiser arm is constructed in a shape that makes deformation of the rigidiser arm in one or more directions difficult, but the rigidiser arm is flexible in at least one direction.

In certain forms, the rigidiser arm has a length that is significantly greater than its width, and a width that is significantly greater than its height.

In certain forms, the rigidiser arms are formed with a curved shape to substantially correspond to the shape of respective sides of the patient's head.

In certain forms, each rigidiser arm is curved when viewed from a direction perpendicular to one of the surfaces of the rigidiser arm facing towards or away from the patient's face in use.

In certain forms, each rigidiser arm curves upwardly so that the superior edge of the rigidiser arm is concave and the inferior edge of the rigidiser arm is convex.

According to one aspect of the present technology, there is provided a positioning and stabilising structure for a patient interface configured to deliver breathable gas at a therapeutic pressure of at least 4 cmH₂O above ambient air pressure to an entrance to a patient's airways. The positioning and stabilising structure may comprise a rear portion configured to overlie a posterior region of the patient's head. The positioning and stabilising structure may further comprise first and second side portions, each being configured, in use, to extend across a respective cheek region of the patient's head, and each having a posterior end connected to a respective side of the rear portion and an anterior end configured to connect directly or indirectly to a seal-forming structure of the patient interface, the seal-forming structure being to form a seal with a region of the patient's face surrounding an entrance to the patient's airways. Each of the first and second side portions may comprise a rigidiser arm positioned inside a sleeve. The first and second side portions may be each tapered such that the posterior end is wider than the anterior end.

In certain forms, the rigidiser arm and the sleeve may be connected together so that substantially no part of the sleeve can move relative to the rigidiser arm.

In certain forms, the sleeve is formed so as to be flexible.

In certain forms, the sleeve is formed from a textile material.

In certain forms, the sleeve may comprise an outer sleeve and an inner sleeve. The inner sleeve may be positioned inside the outer sleeve and the rigidiser arm may be positioned inside the inner sleeve.

In certain forms, the inner sleeve is thicker than the outer sleeve.

In certain forms, the inner sleeve is denser than the outer sleeve.

In certain forms, the inner sleeve is formed with a denser weave or knit pattern than the outer sleeve.

In certain forms, each of the first and second side portions may comprise one or more cushion portions positioned inside the sleeve. For example, each of the first and second side portions may comprise two cushion portions positioned inside the sleeve on opposite lateral sides of the rigidiser arm.

In certain forms, the rigidiser arm may be formed from a heat-activated material. The heat-activated material may be bonded with the sleeve.

In certain forms, the rigidiser arm is constructed in a shape that makes deformation of the rigidiser arm in one or more directions difficult, but the rigidiser arm is flexible in at least one direction.

In certain forms, the rigidiser arm has a length that is significantly greater than its width, and a width that is significantly greater than its height.

In certain forms, the rigidiser arms are formed with a curved shape to substantially correspond to the shape of respective sides of the patient's head.

In certain forms, each rigidiser arm is curved when viewed from a direction perpendicular to one of the surfaces of the rigidiser arm facing towards or away from the patient's face in use.

In certain forms, each rigidiser arm curves upwardly so that the superior edge of the rigidiser arm is concave and the inferior edge of the rigidiser arm is convex.

According to one aspect of the present technology, there is provided a patient interface. The patient interface may comprise a plenum chamber pressurisable to a therapeutic pressure of at least 4 cmH₂O above ambient air pressure. The plenum chamber may include a plenum chamber inlet port sized and structured to receive a flow of air at the therapeutic pressure for breathing by a patient. The patient interface may further comprise a seal-forming structure constructed and arranged to form a seal with a region of the patient's face surrounding an entrance to the patient's airways. The seal-forming structure may have a hole therein such that the flow of air at said therapeutic pressure is delivered to at least an entrance to the patient's nares. The seal-forming structure may be constructed and arranged to maintain said therapeutic pressure in the plenum chamber throughout the patient's respiratory cycle in use. The patient interface may further comprise a vent to allow a continuous flow of gases exhaled by the patient from an interior of the plenum chamber to ambient. The vent may be sized and shaped to maintain the therapeutic pressure in the plenum chamber in use. The patient interface may further comprise a positioning and stabilising structure according to another aspect of the present technology.

According to one aspect of the present technology, there is provided a side strap for a positioning and stabilising structure for a patient interface configured to deliver breathable gas at a therapeutic pressure of at least 4 cmH2O above ambient air pressure to an entrance to a patient's airways. The side strap may comprise a rigidiser arm positioned inside a sleeve. The rigidiser arm and the sleeve may be connected together so that substantially no part of the sleeve can move relative to the rigidiser arm.

In certain forms, the first and second side portions may be each tapered such that the posterior end is wider than the anterior end.

In certain forms, the sleeve is formed so as to be flexible.

In certain forms, the sleeve is formed from a textile material.

In certain forms, the sleeve may comprise an outer sleeve and an inner sleeve, the inner sleeve positioned inside the outer sleeve and the rigidiser arm positioned inside the inner sleeve.

In certain forms, the inner sleeve is thicker than the outer sleeve.

In certain forms, the inner sleeve is denser than the outer sleeve.

In certain forms, the inner sleeve is formed with a denser weave or knit pattern than the outer sleeve.

In certain forms, each of the first and second side portions may comprise one or more cushion portions positioned inside the sleeve. For example, each of the first and second side portions may comprise two cushion portions positioned inside the sleeve on opposite lateral sides of the rigidiser arm.

In certain forms, the rigidiser arm may be formed from a heat-activated material. The heat-activated material may be bonded with the sleeve.

In certain forms, the rigidiser arm is constructed in a shape that makes deformation of the rigidiser arm in one or more directions difficult, but the rigidiser arm is flexible in at least one direction.

In certain forms, the rigidiser arm has a length that is significantly greater than its width, and a width that is significantly greater than its height.

In certain forms, the rigidiser arms are formed with a curved shape to substantially correspond to the shape of respective sides of the patient's head.

In certain forms, each rigidiser arm is curved when viewed from a direction perpendicular to one of the surfaces of the rigidiser arm facing towards or away from the patient's face in use.

In certain forms, each rigidiser arm curves upwardly so that the superior edge of the rigidiser arm is concave and the inferior edge of the rigidiser arm is convex.

According to one aspect of the present technology, there is provided a side strap for a positioning and stabilising structure for a patient interface configured to deliver breathable gas at a therapeutic pressure of at least 4 cmH2O above ambient air pressure to an entrance to a patient's airways. The side strap may comprise a rigidiser arm positioned inside a sleeve. The first and second side portions may be each tapered such that the posterior end is wider than the anterior end.

In certain forms, the rigidiser arm and the sleeve may be connected together so that substantially no part of the sleeve can move relative to the rigidiser arm.

In certain forms, the sleeve is formed so as to be flexible.

In certain forms, the sleeve is formed from a textile material.

In certain forms, the sleeve may comprise an outer sleeve and an inner sleeve, the inner sleeve positioned inside the outer sleeve and the rigidiser arm positioned inside the inner sleeve.

In certain forms, the inner sleeve is thicker than the outer sleeve.

In certain forms, the inner sleeve is denser than the outer sleeve.

In certain forms, the inner sleeve is formed with a denser weave or knit pattern than the outer sleeve.

In certain forms, each of the first and second side portions may comprise one or more cushion portions positioned inside the sleeve. For example, each of the first and second side portions may comprise two cushion portions positioned inside the sleeve on opposite lateral sides of the rigidiser arm.

In certain forms, the rigidiser arm may be formed from a heat-activated material. The heat-activated material may be bonded with the sleeve.

In certain forms, the rigidiser arm is constructed in a shape that makes deformation of the rigidiser arm in one or more directions difficult, but the rigidiser arm is flexible in at least one direction.

In certain forms, the rigidiser arm has a length that is significantly greater than its width, and a width that is significantly greater than its height.

In certain forms, the rigidiser arms are formed with a curved shape to substantially correspond to the shape of respective sides of the patient's head.

In certain forms, each rigidiser arm is curved when viewed from a direction perpendicular to one of the surfaces of the rigidiser arm facing towards or away from the patient's face in use.

In certain forms, each rigidiser arm curves upwardly so that the superior edge of the rigidiser arm is concave and the inferior edge of the rigidiser arm is convex.

Another aspect of one form of the present technology is a patient interface that is moulded or otherwise constructed with a perimeter shape which is complementary to that of an intended wearer.

According to one aspect of the present technology, there is provided a side strap for a positioning and stabilising structure for a patient interface configured to deliver breathable gas at a therapeutic pressure of at least 4 cmH2O above ambient air pressure to an entrance to a patient's airways. The side strap may comprise a rigidiser arm positioned inside a sleeve. The sleeve may comprise an outer sleeve and an inner sleeve, the inner sleeve positioned inside the outer sleeve and the rigidiser arm positioned inside the inner sleeve.

According to one aspect of the present technology, there is provided a side strap for a positioning and stabilising structure for a patient interface configured to deliver breathable gas at a therapeutic pressure of at least 4 cmH2O above ambient air pressure to an entrance to a patient's airways. The side strap may comprise a rigidiser arm positioned inside a sleeve. The side strap may comprise one or more cushion portions positioned inside the sleeve.

According to one aspect of the present technology, there is provided a side strap for a positioning and stabilising structure for a patient interface configured to deliver breathable gas at a therapeutic pressure of at least 4 cmH2O above ambient air pressure to an entrance to a patient's airways. The side strap may comprise a rigidiser arm positioned inside a sleeve. The rigidiser arm may be formed from a heat-activated material, and the heat-activated material may be bonded with the sleeve.

An aspect of one form of the present technology is a method of manufacturing apparatus. In certain forms, there is provided a method of manufacturing a positioning and stabilising structure according to another aspect of the present technology. In certain forms, there is provided a method of manufacturing a patient interface according to another aspect of the present technology.

According to one aspect of the present technology, there is provided a method of forming a side strap for a positioning and stabilising structure for a patient interface. The method may comprise assembling a rigidiser arm and a sleeve with the rigidiser arm positioned inside the sleeve.

According to one aspect of the invention, there is provided a method of forming a side strap for a positioning and stabilising structure for a patient interface, the method comprising assembling a rigidiser arm and a sleeve with the rigidiser arm positioned inside the sleeve, and connecting the rigidiser arm and sleeve together so that substantially no part of the sleeve can move relative to the rigidiser arm.

In certain forms the method further comprises positioning the rigidiser arm inside the sleeve and bonding an outer surface of the rigidiser arm with the inner surface of the sleeve.

In certain forms the method further comprises applying heat to the side strap to cause the outer surface of the rigidiser arm to bond with the inner surface of the sleeve.

In certain forms the method comprises forming the rigidiser arm from a heat-activated material.

In certain forms the method further comprises applying a layer of adhesive to the inner surface of the inner sleeve only, to the outer surface of the rigidiser arm only, or to both the inner surface of the inner sleeve and the outer surface of the rigidiser arm, and positioning the rigidiser arm inside the sleeve.

In certain forms the method further comprises forming the sleeve from an outer sleeve and an inner sleeve, the inner sleeve positioned inside the outer sleeve and the rigidiser arm positioned inside the inner sleeve.

In certain form the method further comprises forming the outer sleeve and the inner sleeve separately, and assembling the outer sleeve and the inner sleeve together to form the sleeve.

In certain forms the method further comprises forming the outer sleeve and the inner sleeve together.

In certain forms the outer sleeve and the inner sleeve are woven or knitted together.

In certain forms the method comprises forming the inner sleeve from a low-melt bonding material.

In certain forms the method further comprises positioning one or more cushion portions inside the sleeve.

In certain forms the method further comprises positioning two cushion portions inside the sleeve on opposite lateral sides of the rigidiser arm.

In certain forms the method further comprises weaving the one or more cushion portions with the sleeve.

In certain forms the method further comprises injecting a fluid substrate into contact with the sleeve and allowing the substrate to harden to form the rigidiser arm.

In certain forms the method further comprises injecting a layer of the substrate onto a first layer of material and positioning a second layer of material in relation to the layer of the substrate such that the layer of the substrate is positioned between the first and second layers of material, wherein the first and second layers of material form the sleeve.

In certain forms the method further comprises holding the sleeve in a predetermined shape and injecting the substrate into the sleeve and allowing the substrate to harden while the sleeve is held in the predetermined shape.

According to one aspect of the technology, there is provided a method of forming a positioning and stabilising structure for a patient interface comprising forming two side straps using any method as described above, and providing the side straps to respective sides of a rear portion of the positioning and stabilising structure configured to overlie a posterior region of the patient's head.

According to one aspect of the present technology, there is provided a method of forming a side strap for a positioning and stabilising structure for a patient interface. The method may comprise assembling a rigidiser arm and a sleeve with the rigidiser arm positioned inside the sleeve. The method may further comprise positioning the rigidiser arm inside the sleeve and bonding an outer surface of the rigidiser arm with the inner surface of the sleeve.

According to one aspect of the present technology, there is provided a method of forming a side strap for a positioning and stabilising structure for a patient interface. The method may comprise assembling a rigidiser arm and a sleeve with the rigidiser arm positioned inside the sleeve. The method may further comprise forming the sleeve from an outer sleeve and an inner sleeve, the inner sleeve positioned inside the outer sleeve and the rigidiser arm positioned inside the inner sleeve.

According to one aspect of the present technology, there is provided a method of forming a side strap for a positioning and stabilising structure for a patient interface. The method may comprise assembling a rigidiser arm and a sleeve with the sleeve positioned inside the rigidiser arm. The method may further comprise injecting a fluid substrate into contact with the sleeve and allowing the substrate to harden to form the rigidiser arm.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, c.g., in soapy water, without requiring specialised cleaning equipment.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

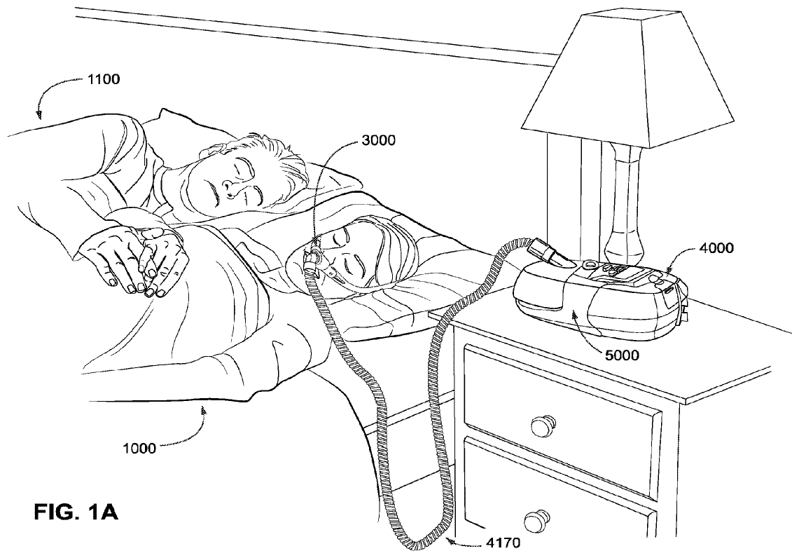

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

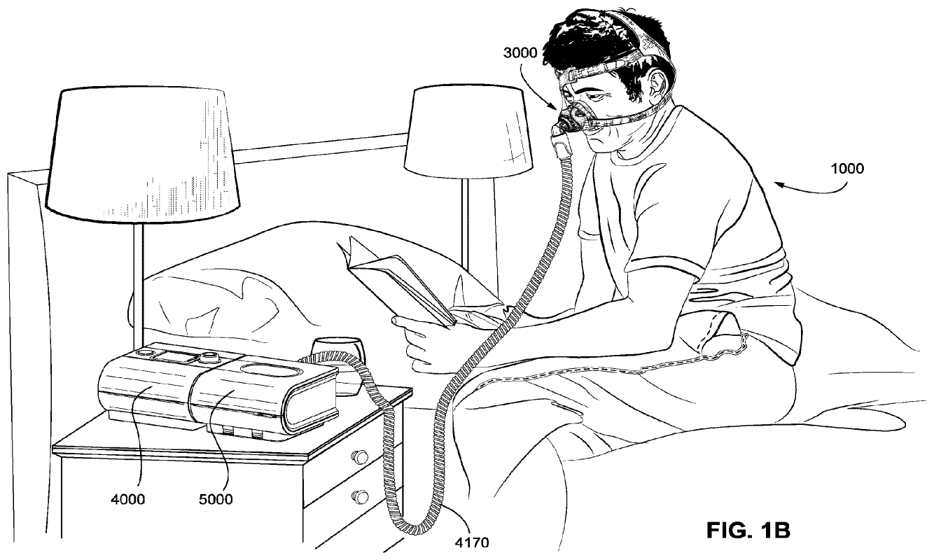

FIG. 2A shows an overview of a human respiratory system including the nasal and oral cavities, the larynx, vocal folds, oesophagus, trachea, bronchus, lung, alvcolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

### 3.3 Patient Interface

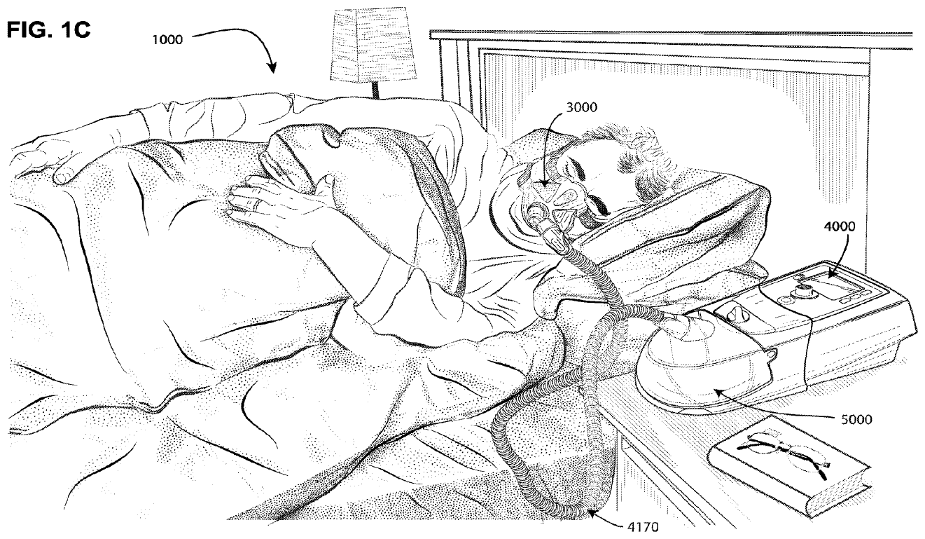

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U.

The direction of the view is normal to the mid-contact plane. The sagittal plane in

FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3201 which lies on the sagittal plane and just touches the cushion of the plenum chamber at two points on the sagittal plane: a superior point 3221 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3221 sits approximately on the sellion, while the inferior point 3230 sits on the lip superior.

FIG. 3Y shows a view of a patient interface 3000 in accordance with one form of the technology.

FIG. 3Z shows a schematic view of a side portion 3320 in accordance with one form of the technology.

FIG. 3AA shows a perspective cross-sectioned view of part of a side portion 3320 in accordance with one form of the technology.

FIG. 3BB shows a perspective cross-sectioned view of the side portion 3320 shown in FIG. 3AA.

FIG. 3CC shows a schematic cross-sectional view of the side portion 3320 shown in FIG. 3AA.

FIG. 3DD shows a view of a side portion 3320 during manufacture in accordance with one form of the technology.

FIG. 3EE shows a partial cross-section view of the side portion 3320 of FIG. 3DD during a subsequent step in its manufacture.

FIG. 3FF shows a schematic view of a sleeve 3340 in accordance with one form of the technology.

FIG. 3GG shows a cross-sectional view of a side portion 3320 in accordance with one form of the technology.

FIG. 3HH shows another cross-sectional view of the side portion 3320 of FIG. 3GG during a subsequent step in its manufacture.

### 3.4 RPT Device

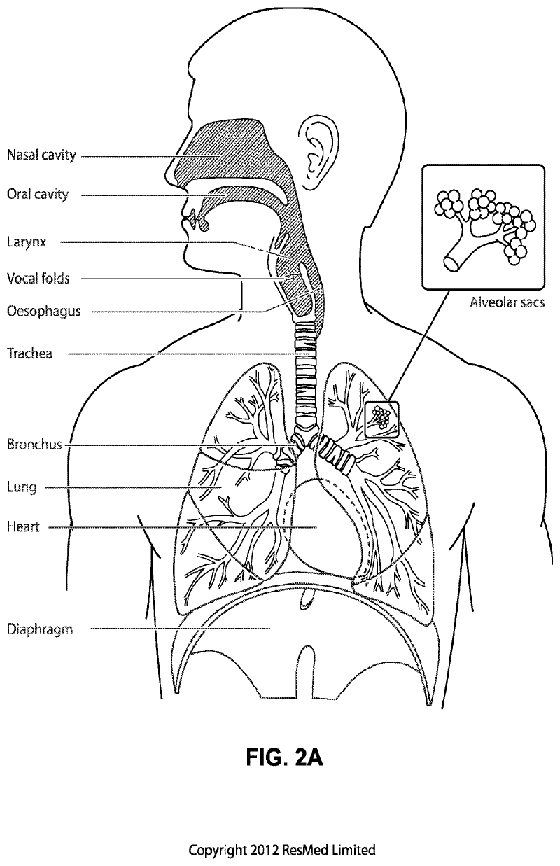

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 Humidifier

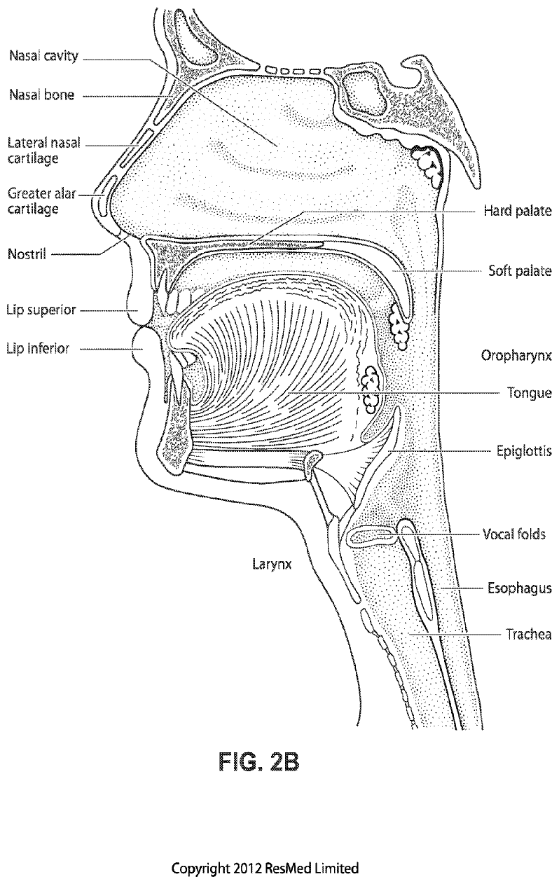

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000, such as that shown in FIG. 3A in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and optionally a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH2 O with respect to ambient.

### 4.3.1 Seal-Forming Structure

The patient interface 3000 may comprise a seal-forming structure 3100. The seal-forming structure 3100 may be constructed and arranged to form a seal with a region of the patient's face surrounding an entrance to the patient's airways. Furthermore, the seal-forming structure 3100 may have a hole therein such that the flow of air at said therapeutic pressure is delivered to at least an entrance to the patient's nares in use. The seal-forming structure 3100 may be constructed and arranged to maintain the therapeutic pressure in the plenum chamber 3200 throughout the patient's respiratory cycle in use.

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs-the actual sealing surface-may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber. In other forms the seal-forming structure 3100 comprises a foam undercushion 3110 and a textile membrane portion 3220, as described further below.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head. However, examples of the technology may be suitable for a large range of heads, and so may be used by patients having a relatively large head and a relatively small head.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight scaling engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In certain forms of the present technology, a seal-forming structure may comprise one or more of a pressure-assisted sealing flange, a compression sealing portion, a gasket sealing portion, a tension portion, and a portion having a tacky or adhesive surface.

### 4.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.5 Forehead Region

In one form, the seal-forming structure that forms a seal in use on a forehead region of the patient's face. In such a form, the plenum chamber may cover the eyes in use.

### 4.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 4.3.1.7 Nose-Only Masks

In certain forms, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 3Y, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's narcs.

In some forms, a nose-only mask may comprise nasal pillows, described above.

### 4.3.1.8 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alac of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 4.3.2 Plenum Chamber

The plenum chamber 3200 may be formed by a portion of the patient interface 3000 that has a perimeter that is shaped to be complementary to the surface contour of the face of an average person in the region where a seal will form in use. In use, a marginal edge of the portion of the patient interface 3000 forming the plenum chamber 3200 is positioned in close proximity to an adjacent surface of the face.

Actual contact with the face is provided by the seal-forming structure 3100. The seal-forming structure 3100 may extend in use about the entire perimeter of a portion of the patient interface 3000 forming the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is formed by one or more components constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is formed by one or more components constructed from a translucent material. The use of a translucent material can reduce the obtrusiveness of the patient interface, and help improve compliance with therapy.

### 4.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head. The multiple positioning and stabilising structures 3300 may be configured to interchangeably connect to other components of the patient interface 3000.

In certain forms, the positioning and stabilising structure 3300 comprises a rear portion 3310 and first and second side portions 3320a and 3320b.

### 4.3.3.1 Rear Portion

The rear portion 3310 of the positioning and stabilising structure 3300 may be configured, in use, to overlie a posterior region of the patient's head. An example rear portion 3310 is shown in FIG. 3Y, in which the rear portion 3310 is worn over a posterior and generally superior part of the patient's head during use.

In certain forms, the rear portion 3310 is bendable and, e.g. non-rigid. An advantage of this is that the rear portion 3310 is able to conform to the shape of the patient's head and may be more comfortable for a patient to lie upon while the patient is sleeping. For example, the rear portion 3310 may comprise one or more straps.

In the example of FIG. 3Y, the rear portion 3310 comprises an inferior posterior strap portion 3312 and a superior posterior strap portion 3314. The inferior posterior strap portion 3312 may be configured to overlie a middle or inferior region of the posterior part of the patient's head during use, for example overlying the occipital bone. The superior posterior strap portion 3314 may be configured to overlie a superior region of the posterior part of the patient's head, for example overlying the parietal bone, and/or may pass substantially over the top of the patient's head when worn. The inferior posterior strap portion 3312 and the superior posterior strap portion 3314 may be formed as two portions of the same length of strap, for example as is the case in the exemplary form shown in FIG. 3Y. Alternatively, the inferior posterior strap portion 3312 and the superior posterior strap portion 3314 may be separate strap portions that are joined together, for example through stitching. In some forms, the inferior posterior strap portion 3312 and the superior posterior strap portion 3314 may be formed as the bifurcated sections of a single strap.

In alternative forms, the rear portion 3310 may comprise a single strap. Alternatively, the rear portion 3310 may comprise a sheet of fabric or textile, a mesh structure, or some other structure configured to sit on or around a posterior region of the patient's head.

In certain forms of the present technology, the rear portion 3310 may comprise at least one strap constructed to be breathable to allow moisture vapour to be transmitted through the strap.

In certain forms of the present technology, the rear portion 3310 may comprise at least one strap constructed from a laminate of a fabric patient-contacting layer, a foam inner layer and a fabric outer layer. In one form, the foam is porous to allow moisture, (e.g., sweat), to pass through the strap.

In certain forms of the present technology, the rear portion 3310 may comprise at least one strap that is extensible, e.g. resiliently extensible. For example the strap may be configured in use to be in tension, and to direct a force to draw the seal-forming structure 3100 into sealing contact with a portion of a patient's face.

### 4.3.3.2 Side Portions

The positioning and stabilising structure 3300 may also comprise first and second side portions 3320, each of which are configured to extend across a respective check region of the patient's head when the patient interface 3000 is being worn. The first and second side portions 3320 may extend between the rear portion 3310 and the part of the patient interface 3000 at the front of the patient's face. That is, a posterior end of each of the first and second side portions 3320 may connect to a respective side of the rear portion 3310 and an anterior end of each of the first and second side portions 3320 may connect directly or indirectly to the seal-forming structure 3100.

As such, the side portions 3320 may act to transmit a force on the seal-forming structure 3100 towards the patients face, i.e. in a generally posterior direction, in order to maintain the seal forming structure 3100 in a sealed engagement with the patient's face during use of the patient interface 3000. Consequently, the first and second side portions 3320 may be under tension during use.

In some forms, the anterior ends of the first and second side portions 3320 may connect directly to the seal-forming structure 3100 while, in other forms, such as that shown in FIG. 3A, the anterior ends of the first and second side portions 3320 may connect indirectly to the seal-forming structure 3100. In forms with such an indirect connection, the anterior ends of the first and second side portions 3320 may connect to any other component of the patient interface 3000 in order to transmit a force to the seal-forming structure 3100. That other component may itself be directly or indirectly attached to the seal-forming structure 3100, i.e. there may be a plurality of intermediate components between the anterior ends of the first and second side portions 3320 and the seal-forming structure 3100. In examples, the anterior ends of the first and second side portions 3320 may connect directly to any one or more of: the plenum chamber 3200; a frame or shell; a forehead connector 3700; headgear connectors; headgear arms.

In some forms, the positioning and stabilising structure 3300 may comprise a single side portion 3320 on each side of the patient's head. This is the case for the form of the technology shown in FIG. 3Y. Such a configuration may be particularly used in the case of a nose-only mask, and in particular a “nasal cradle” mask such as is shown in FIG. 3Y, or a nasal pillows mask. In such forms, the positioning and stabilising structure 3300 may comprise first and second side portions 3320 constructed and arranged so that in use at least a portion of an inferior edge of each side portion 3320 passes superior to an otobasion superior of the patient's head. This is the case with the single side portion 3320 on each side of the patient's head in the example of FIG. 3Y. The posterior end of each side portion 3320 may overlay the patient's head superior to, and optionally posterior to, the patient's otobasion superior, in use, for example overlying the temporal bone and/or the parietal bone. In the form shown in FIG. 3Y, the anterior end of each side portion 3320 overlie a cheek region.

In other forms, the positioning and stabilising structure 3300 may comprise multiple side portions 3320 on each side of the patient's head. For example, in the form shown in FIG. 3A, each side of the positioning and stabilising structure 3300 comprises a superior side portion and an inferior side portion. This arrangement may be particularly suitable for a nasal mask or for a full-face mask. The superior side portions may be configured to pass superior to an otobasion superior of the patient's head when the patient interface 3000 is worn, while the additional side portions may be constructed and arranged so that in use at least a portion of a superior edge thereof passes inferior to an otobasion inferior of the patient's head and overlays or lies inferior to the occipital bone of the patient's head.

In certain forms of the technology, for example as shown in FIGS. 3Y to 3CC, each of the side portions 3320 may comprise a rigidiser arm 3330 and one or more sleeves 3340. These components are described in more detail below.

In certain forms of the technology, for example as shown in FIGS. 3Y to 3CC, each of the side portions 3320 may comprise one or more straps. In such forms, the side portions 3320 may be referred to as side straps.

### 4.3.3.2.1 Shape of Side Portion

As shown in the examples of FIG. 3A, the side portions 3320 are generally elongate members with a length significantly longer than their width or height. In addition, in some forms, including those shown in FIG. 3A, the width of the side portions 3320 may be significantly longer than their height. These relative sizes of the dimensions of the side portions 3320 is the case for side portions formed of straps, as shown in FIG. 3A, or strap-like members, as shown in FIG. 3Y.

For the sake of clarity, it should be understood that, as the terms “length”, “width” and “height” are used in relation to side portions 3320 in this specification, the length of the side portions 3320 is their size in the generally anterior-posterior direction in which the side portions 3320 extend between the rear portion 3310 and the part of the patient interface 3000 positioned in front of the patient's face. The width of the side portions 3320 is their size in the direction that is generally perpendicular to the length and also generally parallel to the patient's skin when the patient interface 3000 is being worn. The height of the side portions 3320 is their size in a direction generally perpendicular to the length and the width, i.e. generally perpendicular to the patient's skin when the patient interface 100 is being worn.

In certain forms of the technology, for example in the form shown in FIG. 3Y, the width of the side portions 3320 may vary along their length. In certain forms, the width may taper along their length from a wider end to a narrower end. In the form shown in FIG. 3Y, the wider end is the posterior end of the side portions 3320 that connects to the rear portion 3310 and the narrower end is the anterior end of the side portions 3320 that connects to the front portion of the patient interface 3000. One advantage of tapering the side portions 3320 in this direction is that the amount of material towards the anterior part of the patient interface 3000 is reduced when compared to a wider anterior part of the side portions 3320, which may provide more comfort and less obstruction to the patient, while the broader posterior end of the side portions 3320 provides stability and strength in regions of the positioning and stabilising structure 3300 where the patient interface 3000 is anchored to the patient's head, e.g. where the posterior regions of the side portions 3320 overlie side regions of the skull.

In other forms, the width of the side portions 3320 may vary in an alternative manner along their length. For example the side portions 3320 may taper from a broader anterior end to a narrower posterior end. Alternatively, the side portions 3320 may have one end broader than the end (with the broader end being either the anterior or posterior end) and a more abrupt change of width somewhere along its length. Alternatively, some forms may have a more complex variation of width along the length of the side portions 3320.

The side edges of the side portions 3320 may be curved and/or soft in order to minimize sharp edges that could cause patient discomfort when wearing the patient interface 3000. This may be achieved through the use of a textile sleeve or other soft material sleeve as an outer layer of the side portions 3320, as described below.

### 4.3.3.2.2 Rigidiser Arm

In certain forms of the technology, each side portion 3320 comprises a rigidiser arm 3330. The rigidiser arm 3330 may be an elongate member that provides an increased level of rigidity to the respective side portion 3320 compared to some other portions of the positioning and stabilising structure 3300.

The rigidiser arm 3330 may be primarily responsible as the part of the side portion 3320 that provides a retaining force to maintain the seal-forming structure 3100 in the desired position on the patient's face. In addition, the rigidiser arm 3330 may be the only rigid component of which the respective side portion 3320 is comprised. Consequently, the shape of the side portion 3320 may be largely governed by the shape of the rigidiser arm 3330. Consequently, the description of the shape of the side portions 3320 provided above should be considered to apply to the shape of the rigidiser arms 3330 in certain forms of the technology.

Each rigidiser arm 3330 may have a sufficient length that it extends along all or substantially all of the length of the respective side portion 3320.

In some forms, the rigidiser arm 3330 is constructed so as to be rigid or semi-rigid in at least one direction. For example, the rigidiser arm 3330 may be constructed from a relatively hard material. Additionally, the rigidiser arm 3330 may be constructed in a shape that makes deformation of the rigidiser arm 3330 in one or more directions difficult, but the rigidiser arm 3330 may be flexible in at least one direction. For example, in the case of a rigidiser arm 3330 used in the form shown in FIG. 3Y, the rigidiser arm 3330 may have a length that is significantly greater than its width, and a width that is significantly greater than its height. In such a form, the rigidiser arm 3330 may be flexible about its width and may be inflexible along its length or height. In other words, the rigidiser arm 3330 may be bendable about an axis along the width of the rigidiser arm 3330, but may be unable to bend about an axis perpendicular to the rigidiser arm 3330 or along its length. This may allow an individual patient to adjust the rigidiser arm 3330 in order to better fit their individual head.

The rigidiser arm 3330 may be formed from a material that has a relatively high modulus of elasticity so that the rigidiser arm 3330 is substantially unable to stretch.

The rigidiser arm 3330 may be pre-formed (e.g. pre-molded) into a desired shape in order to fit a patient's head. For example, the rigidiser arms 3320 may be formed (e.g. molded) with a curved shape to substantially correspond to the shape of respective sides of the patient's head (e.g., overlaying the masseter muscle and/or the temporal bone). That is, when viewed from a direction perpendicular to an edge of the rigidiser arm (i.e. looking in the direction of the width of the rigidiser arm), the rigidiser arm 3330 may bend. In certain forms, the rigidiser arm 3330 may be molded in order to conform to a specific patient's head (c.g., the rigidiser arm 3330 is customized).

In certain forms, the rigidiser arm 3330 may be able to bend in this same direction (i.e. perpendicularly towards the patient's face when the patient interface 3000 is worn) and may be configured to remain in the new position after being bent. This may allow a patient adjust the shape of the rigidiser arm 3330 for their specific head and then the rigidiser arm 3330 will keep the desired shape while in use in order to promote patient comfort.

In certain forms, for example, as shown in FIG. 3Y, each rigidiser arm 3330 may additionally or alternatively be curved when viewed from a direction perpendicular to one of the surfaces of the rigidiser arm facing towards or away from the patient's face in use (i.e. looking in the direction of the height of the rigidiser arm). In the form shown in FIG. 3Y the posterior end of the rigidiser arm 3330 forms a larger angle with the Frankfort horizontal (see FIG. 2E) when the patient interface 3000 is worn compared to the angle that the anterior end of the rigidiser arm 3330 forms with the Frankfort horizontal. That is, the rigidiser arm curves upwardly slightly so that the superior edge of the rigidiser arm 3330 is concave and the inferior edge of the rigidiser arm 3330 is convex. This shape may help the side portions 3320 be positioned away from the patient's eyes when the patient interface 3000 is being worn, which may make the patient interface 3000 more comfortable to wear. In addition, the angle of the rigidiser arm 3330 at the anterior end may determine the direction of the force vector acting on the seal-forming structure 3100 and pulling it into sealed contact with the patient's face. Consequently, the angle of the anterior end of the rigidiser arm may be able to be selected to suit the preferred angle of the force vector for the type of mask (e.g. a nasal mask may require a force vector that has a smaller angle with the Frankfort horizontal compared to a nasal cradle mask).

In certain forms, the rigidiser arm 3330 may be formed from any hard plastic, for example polypropylene, nylon or polycarbonate. In other forms, the rigidiser arm 3330 may be formed from a metal, for example stainless steel (including 316L biocompatibility grade stainless steel), or alloy. In other forms, the rigidiser arm 3330 may be formed from a composite material.

In certain forms, the rigidiser arm 3330 may be formed from a heat-activated material. In one example, the rigidiser arm 3330 is formed from a heat-activated polyurethane, although another material may be used in other examples. In some forms, the heat-activated material may be a heat-activated adhesive.

Alternatively, the rigidiser arm 3330 may be formed from a first material (which may be any of the material stated in the previous paragraph, for example), and may be coated with a heat-activated material, such as heat-activated polyurethane. The heat-activated material may be used to bond the rigidiser arm 3330 to the sleeve 3340, as described further below.

### 4.3.3.2.3 Sleeves

In certain forms of the technology, the side portions 3320 each comprise a sleeve 3340. The rigidiser arms 3330 may each be positioned inside the sleeve 3340 so that the sleeves 3340 envelope or wrap around all or part of the outside of each of the rigidiser arms 3330. The sleeves 3340 may extend along all or a substantial part of the length of the side portions 3320.

In certain forms, each of the sleeves 3340 may comprise a plurality of sleeve parts. For example, as depicted schematically in FIGS. 3Z to 3CC, each side portion 3320 may comprise an outer sleeve 3340a and an inner sleeve 3340b. The inner sleeve 3340b may be positioned inside the outer sleeve 3340a and the rigidiser arm 3330 may be positioned inside the inner sleeve 3340b. In other forms, further sleeve layers may be provided.

The sleeves 3340, including at least one of the outer sleeve 3340a and inner sleeve 3340b in forms in which there are multiple sleeves, may be formed so as to be flexible, e.g. they may be formed from a soft material. Consequently, when the sleeves 3340 are positioned around the rigidiser arm 3330, they may adopt the shape of the rigidiser arm 3330, or a similar shape. For example, when the rigidiser arm 3330 is shaped with a taper, the taper is adopted by the sleeves 3340 positioned around the rigidiser arm 3330 to provide the tapered shape of the side portion 3320 described above and illustrated by way of example in FIG. 3Y. Where the rigidiser arm 3330 has another shape in other forms, the sleeves 3340 may also adopt to that shape.

Since the side portions 3320 may come into contact with the patient's face during use, and particularly the patient's cheek region, it may be beneficial in some forms for the outer sleeve 3340a to be formed from a material that is comfortable for the patient when coming into contact with the patient's face. For example, the outer sleeve 3340a may be formed from a material that is relatively soft (e.g. softer than the material used to form the rigidiser arm 3330) and formed to be flexible. For example, the outer sleeve 3340a may be formed from a textile material or other soft material.

For the purposes of this specification, the term “textile material” should be understood to include woven and non-woven textiles, fabrics and other materials formed from a network of fibres. The outer sleeve 3340a may be formed from a napped or unnapped material. The outer sleeve 3340a may be formed from an elastic or inelastic material. In examples, the outer sleeve 3340a may be formed from any one or more of polyamide, copolyimide, polyester, nylon (e.g. low-melt nylon, brushed nylon), yarn (e.g. multifilament, hot melting yarn, thermo-fusible yarn and bonding fibre). In some forms, the outer sleeve 3340a may be formed from a narrow-woven, narrow-warping and/or narrow-circular textile such as a knit, or any thermo-fusible fabric. In other forms, the outer sleeve 3340a may be formed from felt or foam. In some forms, the outer layer 3340a may be formed with a brushed finish on its outer surface to promote comfort when contacting the patient's face when worn.

As already stated, the inner sleeve 3340b may be positioned inside the outer sleeve 3340a and the rigidiser arm 3330 may be positioned inside the inner sleeve 3340b. The inner sleeve 3340b may have the same, or a similar, length as the outer sleeve 3340a. In some forms, the outer sleeve 3340a may cover, or substantially cover, the inner sleeve 3340b so that the inner sleeve 3340b is not visible. The inner sleeve 3340b may be formed from a napped or unnapped material. The inner sleeve 3340b may be formed from an elastic or inelastic material. The inner sleeve 3340b may be formed from a textile material, for example a non-stretch textile, for example woven fabric or brush tricot fabric made of, for example, nylon or polyester. In some forms, the inner sleeve 3340b may be formed from a low-melt yarn such as a low-melt polyester yarn, polyamide yarns (e.g. nylon66), and/or a biocomposite fibre, PP biocomponent, nylon monofilament, polyester monofilament, hotmelt polyester yarn, etc.

In some forms, the inner sleeve 3340b may be thicker than the outer sleeve 3340a. Additionally or alternatively, the inner sleeve 3340b may be denser than the outer sleeve 3340a. For example, the material used to form the inner sleeve 3340b may be denser than the material used to form the outer sleeve 3340a. Additionally or alternatively, the inner sleeve 3340b may be formed with a denser weave or knit pattern than the outer sleeve 3340a. The greater thickness and/or density of the inner sleeve 3340b may be useful to provide a padded underlayer to the sleeve 3340, providing a soft feel and therefore comfort to the patient. Additionally or alternatively, in forms of the technology in which the side portion 3320 comprises a rigidiser arm 3330 positioned inside the sleeve 3340, a thicker inner sleeve 3340b may help to provide protection from the rigidiser arm 3330 contained therein. This protection may amount to protecting the patient from feeling the rigidiser arm 3330 when the patient interface 3000 is being worn and/or it may amount to helping protect from the rigidiser arm 3330 pushing against the outer sleeve 3340a and wearing it thin or possibly poking through the outer sleeve 3340a.

In some forms, the outer sleeve 3340a and the inner sleeve 3340b may be formed separately and then assembled together, for example through stitching or bonding. In other forms, the outer sleeve 3340a and the inner sleeve 3340b may be formed together in the same manufacturing process. For example, the sleeve 3340 comprising both the outer sleeve 3340a and the inner sleeve 3340b may be weaved during a single weaving process. Exemplary machines that may be used to form the sleeve 3340 are a weaving machine, for example a narrow weaving casing machine, and a knitting machine, for example a 4D warp knitting machine.

The rigidiser arm 3330 may be positioned inside the sleeve 3340 (which may comprise both the outer sleeve 3340a and the inner sleeve 3340b, or just one of these) using any suitable technique. In one example, the rigidiser arm 3330 is pushed into the sleeve 3340 when the sleeve 3340 has already been assembled. In forms in which the sleeve 3340 comprises an outer sleeve 3340a and an inner sleeve 3340b, the inner sleeve 3340b may first be inserted into the outer sleeve 3340a, before the rigidiser arm 3330 is inserted into the sleeve assembly. In another such form, the outer sleeve 3340a and inner sleeve 3340b may be assembled together in a different manner, for example using a knitting technique. In another example, the rigidiser arm 3330 is first inserted into the inner sleeve 3340b and the combined rigidiser arm and inner sleeve assembly is subsequently inserted into the outer sleeve 3340a.

In certain forms of the technology, the sleeve 3340 is connected to the rigidiser arm 3330 in such a way that substantially no part of the sleeve 3340 can move relative to the rigidiser arm 3330. In forms in which the sleeve 3340 comprises the outer sleeve 3340a and the inner sleeve 3340b, the outer sleeve 3340a and the inner sleeve 3340b may further be connected together so that substantially no part of the inner and outer sleeves may move relative to each other. In this way, the rigidiser arm 3330 and sleeve 3340 (including the outer and inner sleeves, if present) may form an integrated component. This configuration may provide a more comfortable experience for the user. For example, compared to other patient interfaces which don't have integrated rigidiser arms and sleeves, forms of the present technology may be more comfortable when the patient interface 3000 contacts the patient's skin, may fit the patient's face more comfortably and stably, and may help ensure clearance of components from around the eyes during use, avoiding the patient's vision being impeded and further providing comfort. In contrast, with a patient interface where a sleeve is positioned loosely on a rigidiser, for example, the sleeve may move against the patient's face into an undesired position, which may be uncomfortable, may result in the patient interface being incorrectly positioned, or may encroach on the patient's vision. These advantages consequently help improve the fit of the patient interface, which improves the quality of seal formed by the seal-forming structure 3100 and ultimately helps improve the effectiveness of therapy.

In different forms of the technology, different techniques may be used to achieve this integrated connection between the sleeve 3340 (and its sub-parts, if present) and the rigidiser arm 3330. In some forms, an adhesive may be used to adhere the sleeve 3340 to the rigidiser arm 3330, for example the layer of adhesive 3360 shown in FIG. 3CC. Any suitable adhesive may be used, for example hot-melt, powder, reactive liquids, solvent and waterborne adhesives. In such forms, the layer of adhesive 3360 may adhere the outer surface of the rigidiser arm 3330 to the inner surface of the sleeve 3340 (for example the inner surface of the inner sleeve 3340b, if present). To assemble the side portion 3320 in such forms, the adhesive may be applied to the inner surface of the inner sleeve 3340b only, to the outer surface of the rigidiser arm 3330 only, or to both the inner surface of the inner sleeve 3340b and the outer surface of the rigidiser arm 3330. In other forms, the sleeve 3340 may be thermally bonded to the rigidiser arm 3330, for example any of the following thermally bonding techniques may be used: radiant heat, through-air, ultrasonic, flame, extrusion, calendering, hot bar or iron, hot wedge or laser.

In certain forms, for example the form of the technology shown in FIG. 3AA to 3CC, the rigidiser arm 3330 may be formed from a heat-activated material (either entirely or in part as described earlier, for example a coating of the rigidiser arm may be formed from a heat-activated material). Activation of the material once the rigidiser arm 3330 has been inserted into the sleeve 3340 may involve applying heat to the side portion 3320. The effect of the application of heat may be to alter the state of the heat-activated material so that it is able to adhere or otherwise bond with adjacent materials. Consequently, the outer surface of the rigidiser arm 3330 may be able to bond with the inner surface of the sleeve 3340.

In forms of the technology in which an adhesive and/or heat-activated material is used to bond the rigidiser arm 3330 to the sleeve 3340, it may be advantageous for the sleeve 3340 to be formed in a way that prevents material, for example the adhesive or the heat-activated material, from seeping through to the surface of the sleeve 3340 where it may contact the patient during use (which may be uncomfortable) or be seen (and affect the visual appeal of the side portions 3320). This may be achieved by providing the sleeve 3340 in a variety of ways, for example providing the sleeve 3340 with sufficient density of weave to prevent seepage through it, providing the sleeve 3340 with sufficient thickness to prevent seepage through it, and providing the sleeve 3340 with a relatively thick and/or dense inner sleeve 3340b to substantially prevent seepage through to the outer sleeve 3340a.

In other forms, the sleeve 3340 may be mechanically bonded to the rigidiser arm 3330, for example using pressure embossing. In other forms, a combination of two or more of the aforementioned techniques may be used.

In some forms of the technology, the side portions 3320 may comprise an outer sleeve 3340a, inner sleeve 3340b and rigidiser arm 3330. The inner sleeve 3340b may be formed from a low-melt bonding material, e.g. a low-melt yarn, and a thermal bonding technique may be applied in order to bond the inner sleeve 3340b to both the outer sleeve 3340a and the rigidiser arm 3330. That is, after the outer sleeve 3340a, inner sleeve 3340b and rigidiser arm 3330 have been assembled together, heat is applied at a temperature sufficient to cause the low-melt bonding material to bond with the outer sleeve 3340a and the rigidiser arm 3330.

While it has been described that the sleeve 3340 is connected to the rigidiser arm 3330 in such a way that substantially no part of the sleeve 3340 can move relative to the rigidiser arm 3330, it may be appreciated that, in some forms of the technology, small regions of the sleeve 3340 may be able to move a relatively small amount relative to each other. For example, if an adhesive is used to adhere the sleeve 3340 to the rigidiser arm 3330, there may be regions between these components in which there is no adhesive. In such regions, a small amount of movement between the components may occur. Something similar may occur in the case of other bonding techniques. Nevertheless, in certain forms, the overall effect of the integrated connection between the sleeve 3340 and the rigidiser arm 3330 may be to substantially prevent movement of substantially all parts of one of these components relative to substantially all parts of the other component. The same remarks apply to both the outer sleeve 3340a and the inner sleeve 3340b, if both arc present.

In some forms of the technology, for example as shown in FIG. 3AA to 3CC, the side portion 3320 may comprise one or more cushion portions 3350. The cushion portions 3350 may be positioned inside the sleeve 3340. For example, the cushion portions 3350 may be positioned inside the sleeve 3340 on opposite lateral sides of the rigidiser arm 3330. In this case, a “lateral side” should be understand as referring to a side of the side portion 3320 in the direction of the width of the side portion 3320, as defined earlier.

In certain forms, the cushion portions 3350 provide bulk to the side portions 3320 where the rigidiser arm 3330 is narrower than the width of the sleeve 3340 and therefore, without the cushion portions 3350, the inner circumference of the sleeve 3340 would be greater than the outer circumference of the rigidiser arm 3330. This might lead to rumpling of the material used for the sleeve 3340, which might make the positioning and stabilising structure 3300 uncomfortable to wear. The cushion portions 3350 may additionally or alternatively help to provide a softness to the side portions 3320, especially at the lateral edges of the side portions 3320, where straps can traditionally lead to discomfort and facial marking.

The cushion portions 3350 may be formed from a material that has sufficient resilience to enable the side portion 3320 to substantially retain its shape. In some forms, the cushion portions 3350 may be formed from nylon, polyester and/or polyethylene terephthalate (PET). The material used to form the cushion portions 3350 may be considered to be fibrefill, in some forms. In view of the cushion portions 3350 extending lengthwise along the side portions 3320, and therefore having a length that is significantly larger than their width or height, the cushion portions 3350 may be referred to as filaments.

In some forms of the technology, the cushion portions 3350 may be formed along with the sleeve 3340 as part of the same process. For example, in forms where a weaving process is used to form the sleeve 3340, the cushion portions 3350 may be formed during the same weaving process. It will be apparent that, in such a case, the cushion portions 3350 may be integrally formed with the sleeve 3340. In certain forms, for example as shown in FIG. 3CC, the cushion portions may comprise weave that spans across the side portion 3320 between the inner surfaces of the patient-facing and non-patient-facing sides of the sleeve 3340. In alternative forms, the cushion portions 3350 may be formed separately to the sleeve 3340 and inserted into the sleeve 3340 during assembly of the side portions 3320.

In some forms of the technology, for example as shown in FIG. 3DD to 3HH, the side portions 3320 may be formed by allowing a fluid substrate to harden to form the rigidiser arm 3330. The fluid substrate may be injected into contact with the sleeve 3340 and subsequently allowed to harden. In some forms, the hardening may amount to a curing of the substrate material.

A first step of an exemplary method in such a form of the technology is illustrated in FIG. 3DD. In this form, a fluid substrate may be injected into contact with a first layer of material 3342. The first layer of material 3342 may be a strip of material with a length significantly longer than its width and its width significantly longer than its height. The first layer of material 3342 will ultimately form part of the sleeve 3340 and consequently it have a shape that is the intended shape of the sleeve 3340, for example any of the shapes for the sleeve described earlier. A fluid substrate may be brought into contact with the first layer of material 3342. For example, the fluid substrate may be injected onto a first side of the first layer of material 3342 to form a layer of substrate 3332. The layer of substrate 3332 may substantially cover, e.g. cover a substantial portion of, the first side of the first layer of material 3342. The shape of the layer of substrate 3332 may substantially correspond to the shape of the first side of the first layer of material 3342. In the illustrated form of FIG. 3DD this shape is a long rectangle but this is for illustrative purposes only and it may be other shapes in other forms of the technology. The first side of the first layer of material 3342 may be a patient-facing side or a non-patient-facing side of the first layer of material 3342 when the side portion 3320 is in use. In some forms, the first layer of material 3342 may have a similar texture on both sides. In other forms, the texture may vary on each side, in which case the layer of substrate 3332 may be injected onto the side of the first layer of material 3342 that is the least fluffy or the least soft so that, in use, the fluffiest/softest side may be presented to the patient's face for comfort. This side may be the technical face or the technical back of the first layer of material 3342.

In a subsequent step, as shown in schematic form in FIG. 3EE, a second layer of material 3344 may be positioned in relation to the layer of the substrate 3332 such that the layer of substrate 3332 is positioned between the first and second layers of material 3342 and 3344. As shown in the illustrated example, the second layer of material 3344 may be sized and shaped substantially similarly to the first layer of material 3342. The second layer of material 3344 may be able to cover a substantially part, or all, of the layer of substrate 3332 (although the side portion 3320 is shown in partial cross-section in FIG. 3EE so some of the layer of substrate 3332 is visible in this illustration). In some forms, the edge regions of the first and second layers of material 3342 and 3344 may be joined so that together the first and second layers of material 3342 and 3344 form the sleeve 3340. For example, the edge regions may be stitched, thermoformed or heat bonded together. Similarly to the first layer of material 3342, the second layer of material 3344 may be oriented so that the fluffier/softer side, if there is one, is facing outwardly from the side portion 3320 for the advantage of patient comfort when the side portion 3320 is in use.

The second layer of material 3344 may be positioned in relation to the layer of substrate 3332 before the layer of substrate 3332 is allowed to harden. After positioning the second layer of material 3344 in place, the layer of substrate 3332 is then allowed to harden. Consequently, the layer of substrate 3332 forms a rigid or semi-rigid element, i.e. the rigidiser arm 3330 such as described earlier. In hardening while the layer of substrate 3332 is in contact with the first and second layers of material 3342 and 3344, this allows the rigidiser arm 3330 to bond with the sleeve 3340 because the substrate material forming the rigidiser arm 3330 is able to seep between or around parts of the sleeve 3340 and consequently connect to it across the respective surfaces of the components.

Another form of side portion 3320 according to the technology is illustrated in FIG. 3FF to 3HH. In FIG. 3FF there is provided a sleeve 3340 which may be a hollow tube of material, for example textile. The sleeve 3340 may have a natural shape that is substantially cylindrical, such as shown in FIG. 3FF, or the sleeve 3340 may have some other natural shape. In this context, “natural shape” refers to the shape of the object when no forces that distort the shape of the object are imparted on the object. For example, the natural shape of the sleeve 3340 may be the intended shape of the side portion 3320. In other forms, the rigidiser arm 3330 may, when positioned inside the sleeve 3340, distort the natural shape of the sleeve 3340 so that the assembled side portion 3320 adopts the intended shape as determined by the shape of the rigidiser arm 3330.

The side portion 3320 may be formed by injecting a substrate into the sleeve 3340, for example by injecting a fluid substrate into the sleeve 3340. In some forms, the fluid substrate may be injected into the sleeve 3340 and permitted to adopt the natural shape of the sleeve 3340. In other forms, the sleeve 3340 may be held in a predetermined shape and the fluid substrate may be injected into the sleeve 3340 while the sleeve 3340 is being held in the predetermined shape. A jig may be used to hold the sleeve 3340 in the desired shape and the fluid substrate may be injected into he sleeve 3340 while it is held in the jig. The jig may comprise a hollow chamber whose inner surface has a shape similar to that which the side portion 3320 is intended to have. For example, in the case of the side portion 3320 illustrated in FIG. 3GG and 3HH, the jig may have an inner surface with a cross-sectional area that is pill-shaped. Once assembled, as shown in FIG. 3GG and 3HH, this form is similar in overall cross-sectional shape to the form shown in FIG. 3CC. In both forms, the shape of the outline of the side portion 3320 in cross-section may be considered pill-shaped, “stadium-shaped” or discorectangular.

After injection, the fluid substrate may be allowed to harden so as to form rigidiser arm 3330. As described in relation to other forms, the hardening of the rigidiser arm 3330 may cause the rigidiser arm 3330 to be bonded or otherwise connected with the sleeve 3340 and, in some forms, the rigidiser arm 3330 and the sleeve 3340 may be therefore connected together so that substantially no part of the sleeve 3340 can move relative to the rigidiser arm 3330.

In the forms of the technology shown in FIG. 3DD to 3HH and as described above, the material used to form the first and second layers of material 3342 and 3344 may be any of the materials described earlier in relation to the sleeve 3340 or parts thereof. The material used to form the fluid substrate may be a material that may be injected in fluid form and allowed to harden, for example silicone rubber, rigidising foam, polyester fibrefill and non-woven polypropylene.

In some forms, the thickness of the sleeve 3340 may be in the range of approximately 0.5 mm to approximately 2.5 mm. Additionally, or alternatively, the thickness of the rigidiser arm 3330 may be in the range of approximately 0.5 mm to approximately 3 mm.

### 4.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

In some forms, the patient interface 3000 may comprise a vent 3400 to allow a continuous flow of gases exhaled by the patient from an interior of the plenum chamber 3200 to ambient, for example throughout the patient's entire respiratory cycle, said vent 3400 being sized and shaped to maintain the therapeutic pressure in the plenum chamber in use.

### 4.3.5 Decoupling Structure(s)

In one form the patient interface 3000 includes at least one decoupling structure, for example, a swivel or a ball and socket.

### 4.3.6 Connection Port

Connection port 3600 allows for connection to the air circuit 4170.

### 4.3.7 Forehead Support

In the form shown in FIG. 3A, the patient interface 3000 includes a forehead support 3700.

### 4.3.8 Anti-Asphyxia Valve

In one form, the patient interface 3000 includes an anti-asphyxia valve.

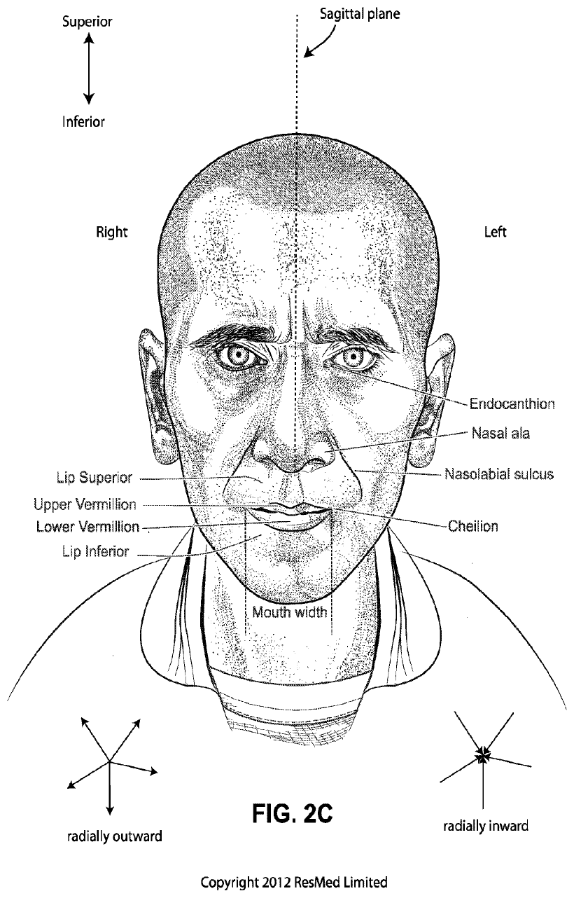

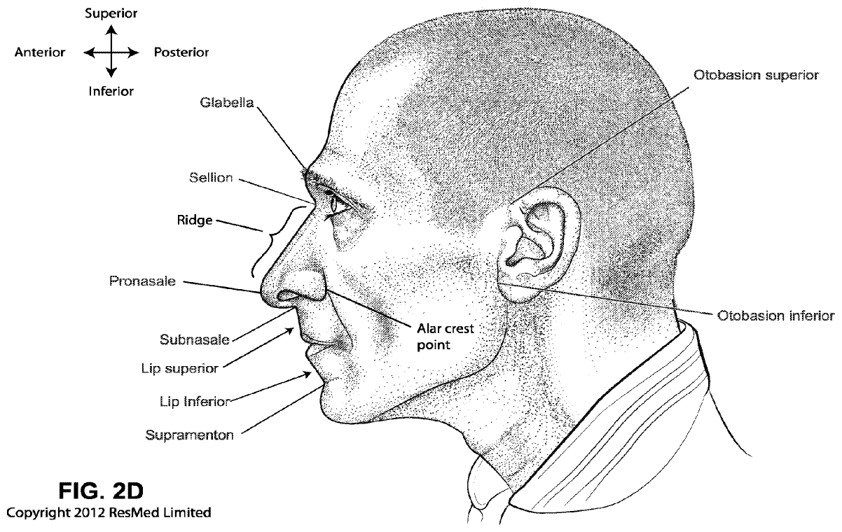

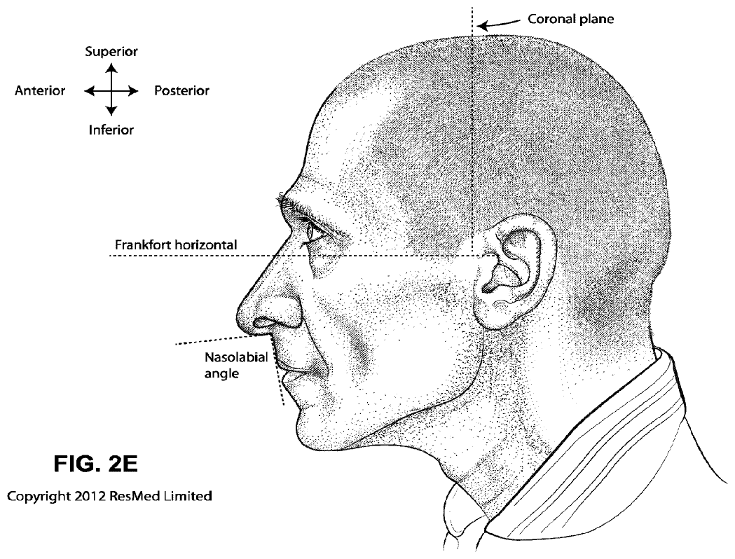

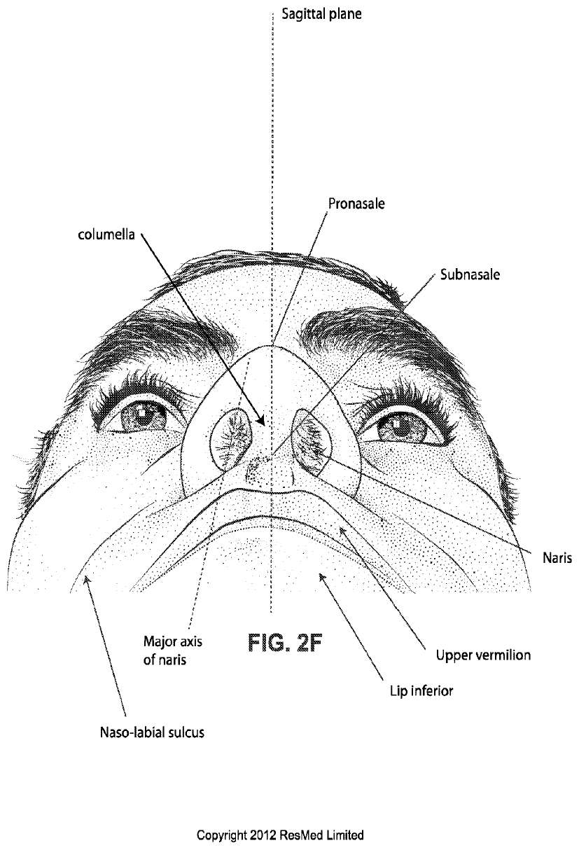

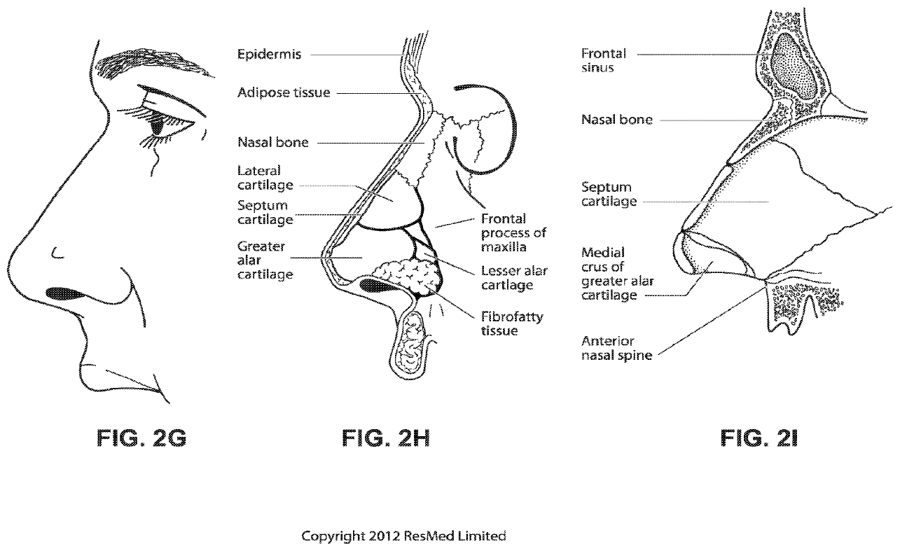

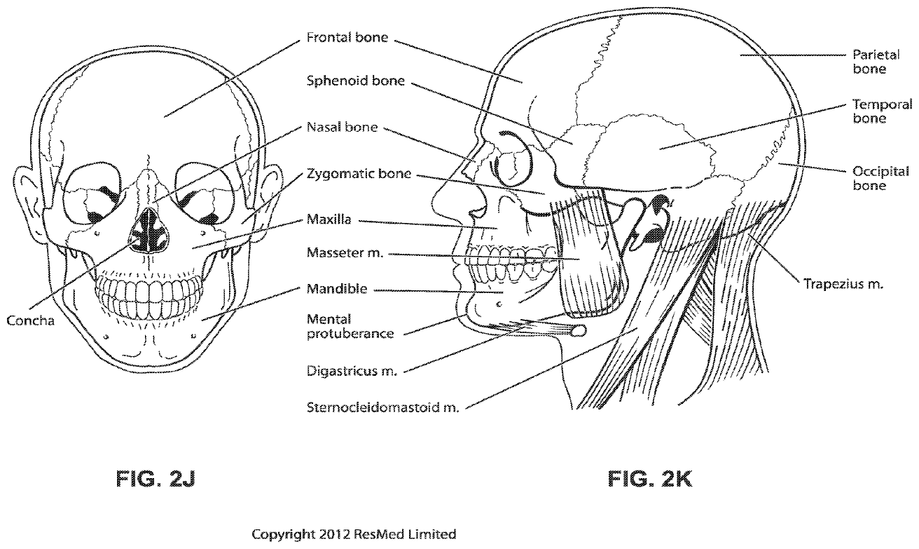

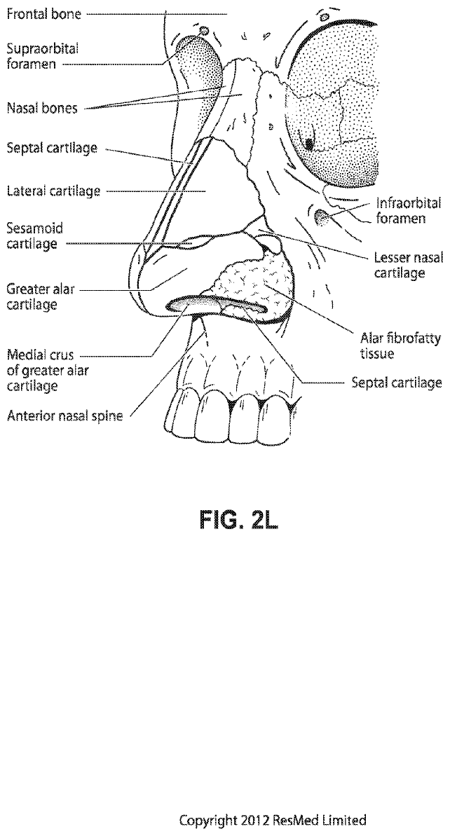

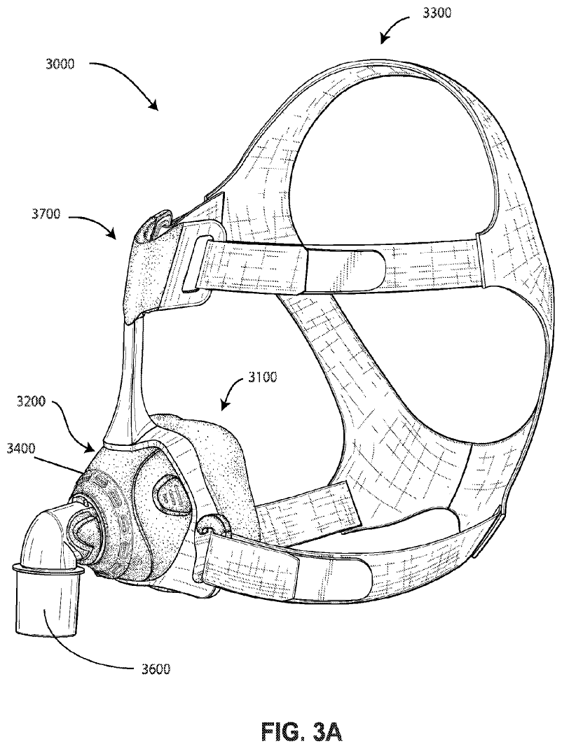

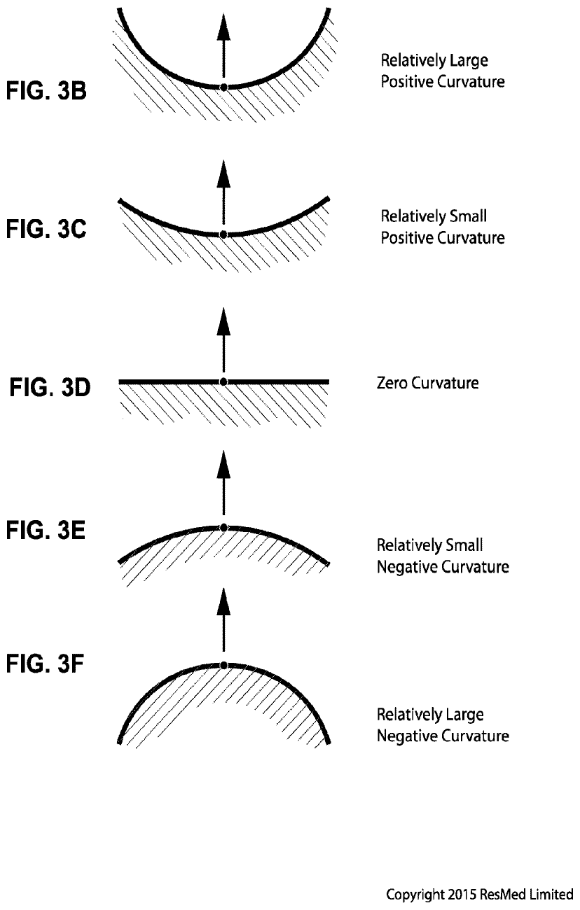

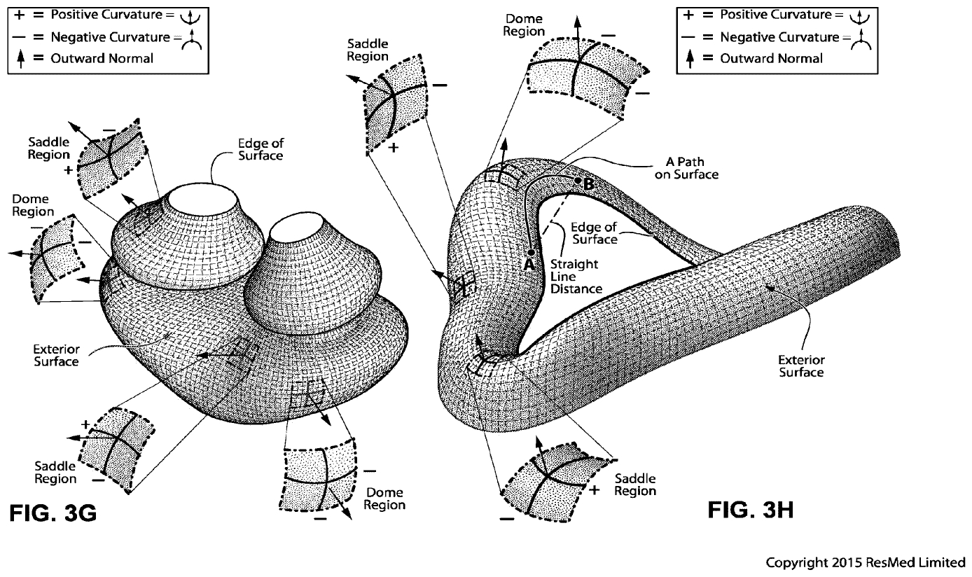

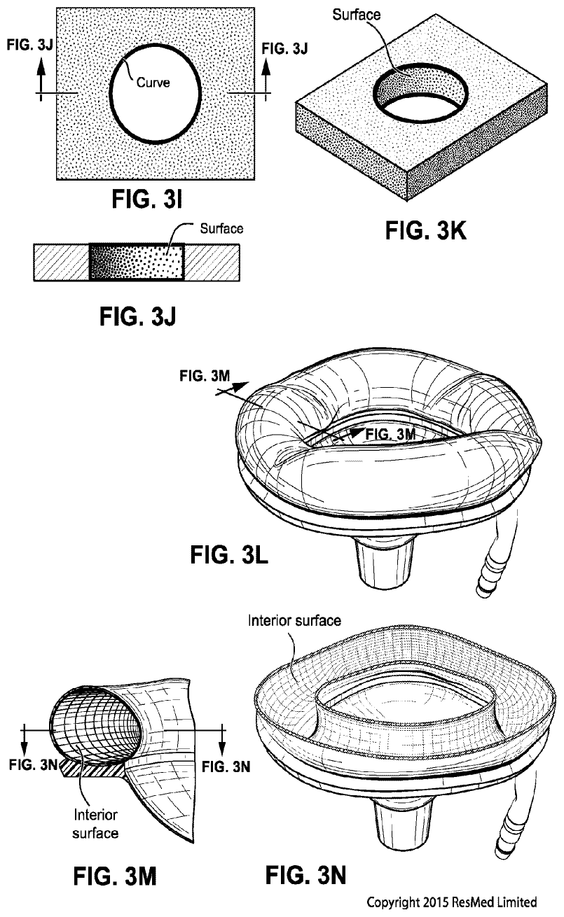

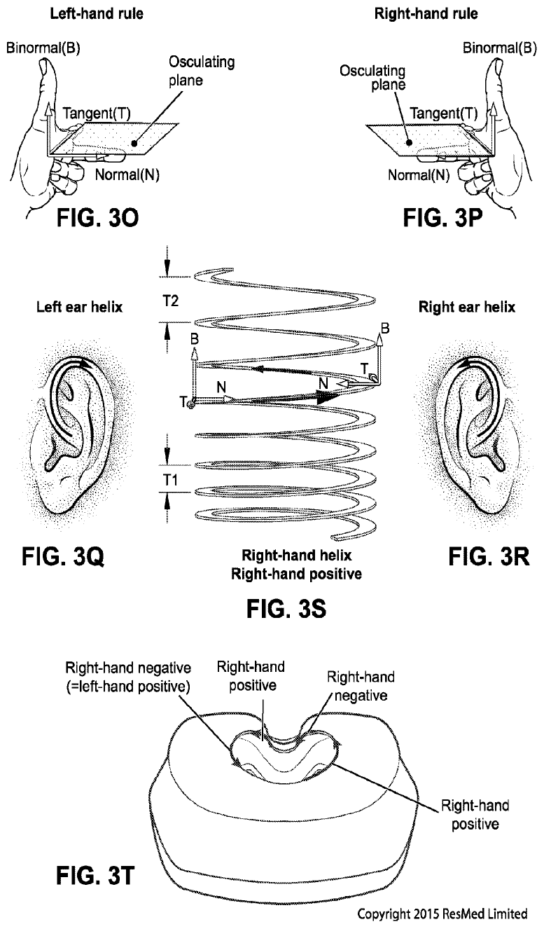

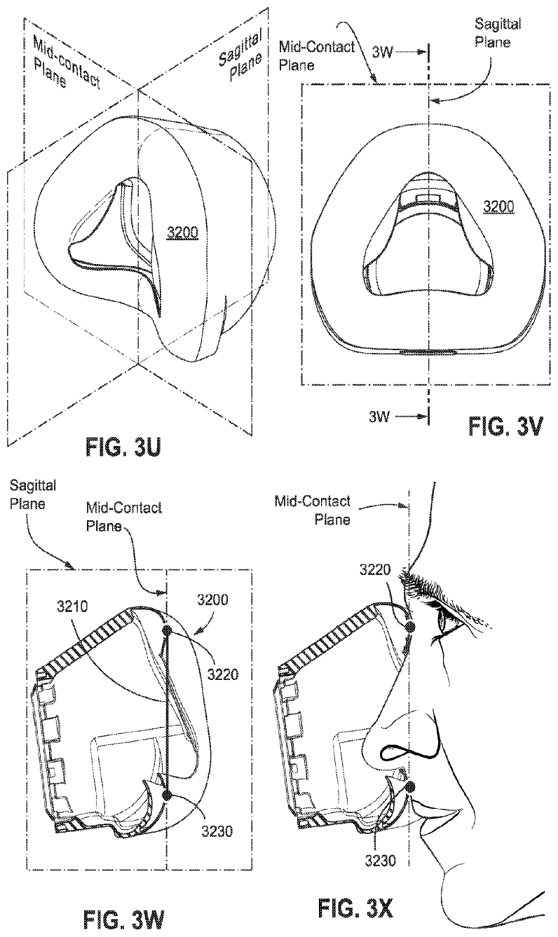

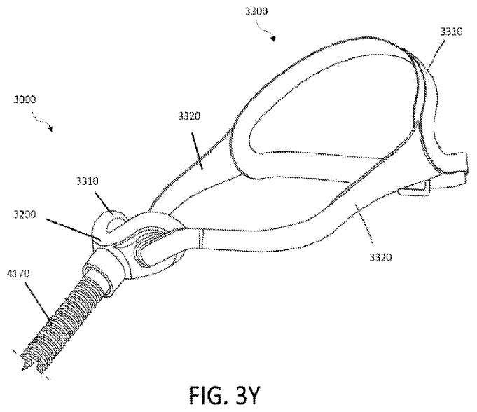

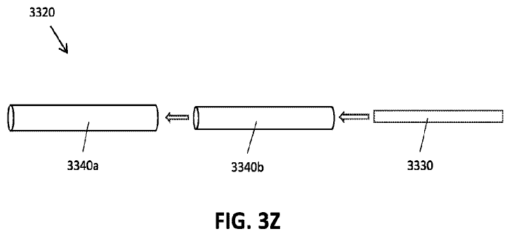

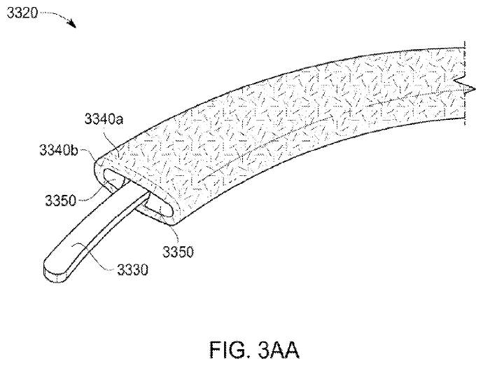

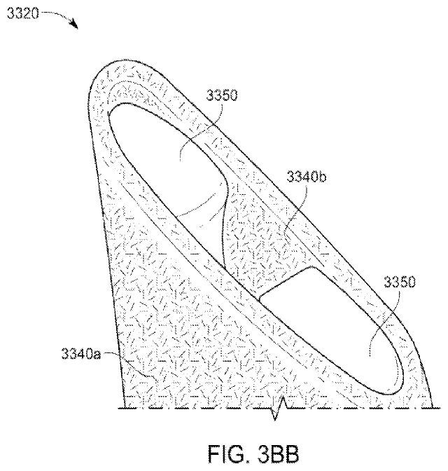

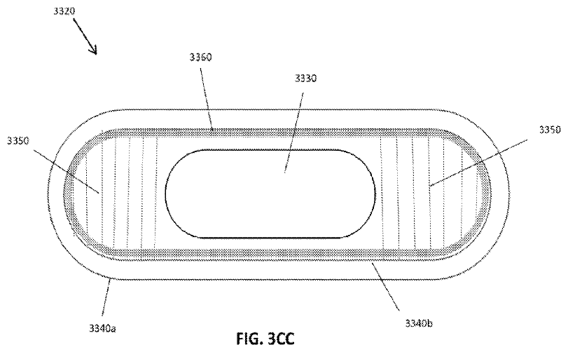

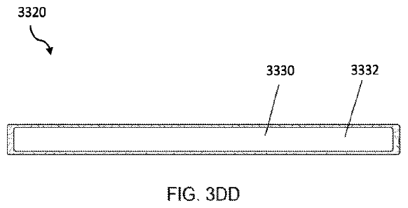

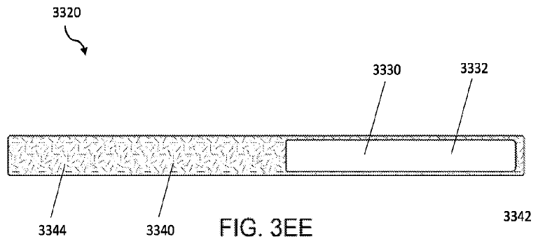

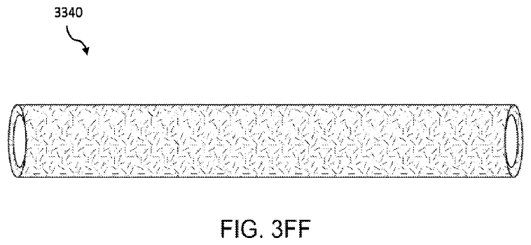

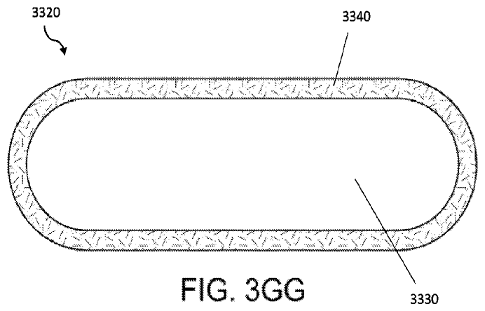

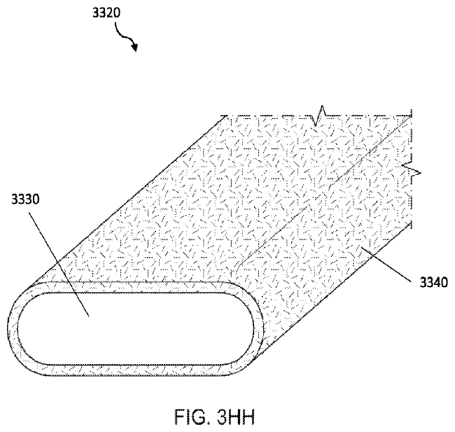

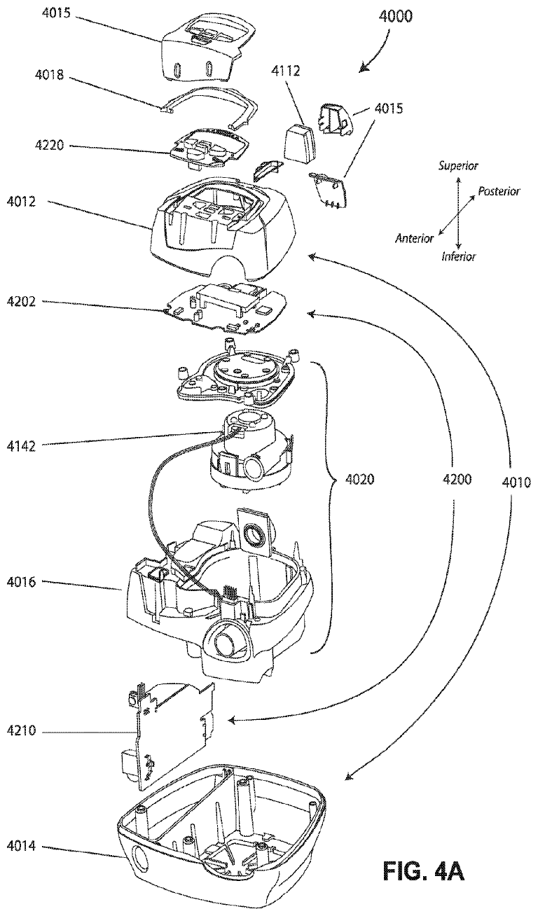

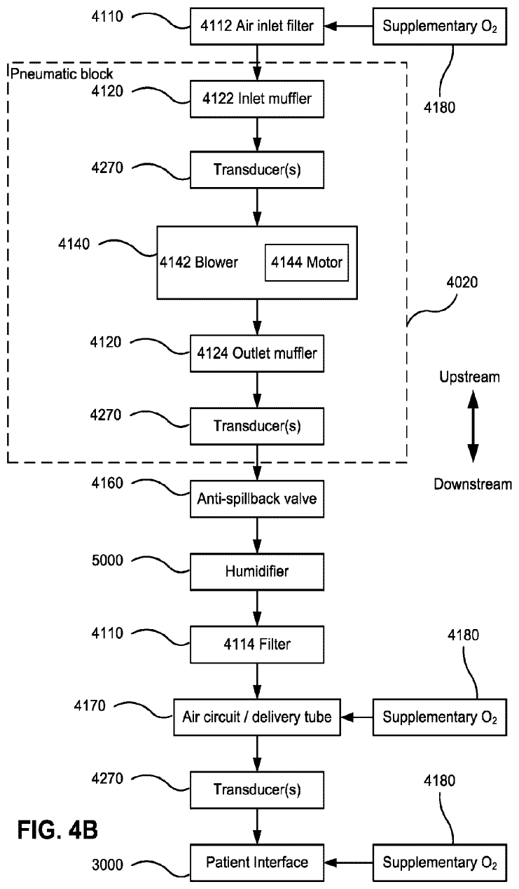

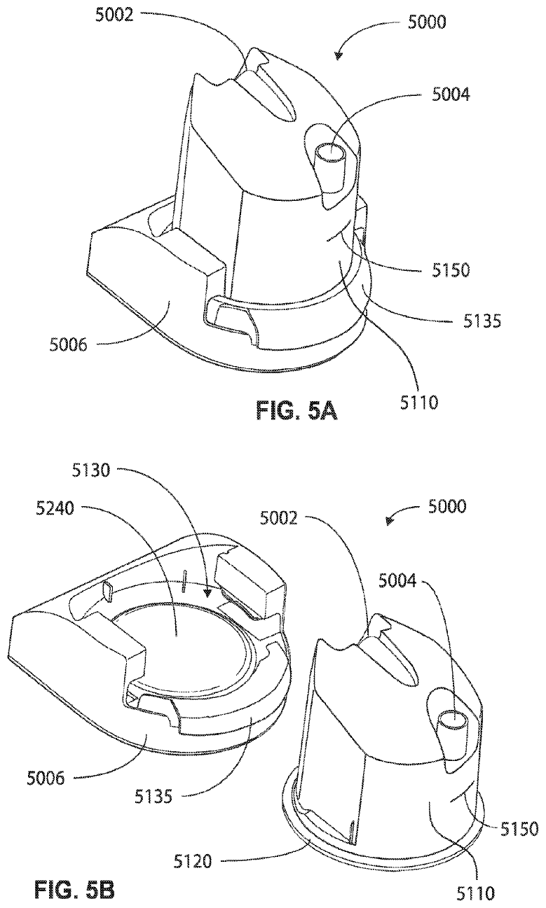

The patient interfaces 3000 shown in or described with reference to FIGS. 18-43 each further comprise an anti-asphyxia valve (AAV), not visible in the drawings. The AAV may be provided to the oral portion of the patient interface 3000. In some examples the AAV is integrated into the vent module 3410. The vent module 3410 may comprise a gas washout vent 3400 and an AAV. In some examples the AAV is provided in the chassis portion 3210 (or oral chassis portion 3217) of the patient interface 3000. In further examples the AAV is provided at a connection port 3600 of the patient interface 3000 or a connection between the plenum chamber 3200 and a short tube 3610, such as an inlet port connector.

### 4.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.4 RPT Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH2O, or at least 10 cmH2O, or at least 20 cmH2O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors and flow rate sensors.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller, a therapy device controller, a pressure generator 4140, one or more protection circuits, memory, transducers 4270, a data communication interface and one or more output devices. Electrical components 4200 may be mounted on a single Printed Circuit Board Assembly (PCBA) 4202. In an alternative form, the RPT device 4000 may include more than one PCBA 4202.

### 4.5 Air Circuit

An air circuit 4170 in accordance with an aspect of the present technology is a conduit or a tube constructed and arranged to allow, in use, a flow of air to travel between two components such as RPT device 4000 and the patient interface 3000.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

### 4.6 Humidifier

### 4.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 4.7 Glossary

For the purposes of the present technology disclosure, in certain forms of the present technology, one or more of the following definitions may apply. In other forms of the present technology, alternative definitions may apply.

### 4.7.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (c.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar~0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.7.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 4.7.1.2 Mechanical Properties

Resilience: Ability of a material to absorb energy when deformed elastically and to release the energy upon unloading.

Resilient: Will release substantially all of the energy when unloaded.

Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not c.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, c.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 4.7.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Expiratory portion of a breathing cycle: The period from the start of expiratory flow to the start of inspiratory flow.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(3272) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

### 4.7.3 Anatomy

### 4.7.3.1 Anatomy of the Face

Ala: the external outer wall or “wing” of each nostril (plural: alar)
Alar angle:
Alare: The most lateral point on the nasal ala.
Alar curvature (or alar crest) point: The most posterior point in the curved base line of each ala, found in the crease formed by the union of the ala with the cheek.
Auricle: The whole external visible part of the ear.
(nose) Bony framework: The bony framework of the nose comprises the nasal bones, the frontal process of the maxillae and the nasal part of the frontal bone.
(nose) Cartilaginous framework: The cartilaginous framework of the nose comprises the septal, lateral, major and minor cartilages.
Columella: the strip of skin that separates the nares and which runs from the pronasale to the upper lip.
Columella angle: The angle between the line drawn through the midpoint of the nostril aperture and a line drawn perpendicular to the Frankfort horizontal while intersecting subnasale.
Frankfort horizontal plane: A line extending from the most inferior point of the orbital margin to the left tragion. The tragion is the deepest point in the notch superior to the tragus of the auricle.
Glabella: Located on the soft tissue, the most prominent point in the midsagittal plane of the forehead.
Lateral nasal cartilage: A generally triangular plate of cartilage. Its superior margin is attached to the nasal bone and frontal process of the maxilla, and its inferior margin is connected to the greater alar cartilage.
Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.
Nares (Nostrils): Approximately ellipsoidal apertures forming the entrance to the nasal cavity. The singular form of nares is naris (nostril). The nares are separated by the nasal septum.
Naso-labial sulcus or Naso-labial fold: The skin fold or groove that runs from each side of the nose to the corners of the mouth, separating the cheeks from the upper lip.
Naso-labial angle: The angle between the columella and the upper lip, while intersecting subnasale.
Otobasion inferior: The lowest point of attachment of the auricle to the skin of the face.
Otobasion superior: The highest point of attachment of the auricle to the skin of the face.
Pronasale: the most protruded point or tip of the nose, which can be identified in lateral view of the rest of the portion of the head.
Philtrum: the midline groove that runs from lower border of the nasal septum to the top of the lip in the upper lip region.
Pogonion: Located on the soft tissue, the most anterior midpoint of the chin.
Ridge (nasal): The nasal ridge is the midline prominence of the nose, extending from the Sellion to the Pronasale.
Sagittal plane: A vertical plane that passes from anterior (front) to posterior (rear). The midsagittal plane is a sagittal plane that divides the body into right and left halves.
Sellion: Located on the soft tissue, the most concave point overlying the area of the frontonasal suture.
Septal cartilage (nasal): The nasal septal cartilage forms part of the septum and divides the front part of the nasal cavity.
Subalare: The point at the lower margin of the alar base, where the alar base joins with the skin of the superior (upper) lip.
Subnasal point: Located on the soft tissue, the point at which the columella merges with the upper lip in the midsagittal plane.
Supramenton: The point of greatest concavity in the midline of the lower lip between labrale inferius and soft tissue pogonion

### 4.7.3.2 Anatomy of the Skull

Frontal bone: The frontal bone includes a large vertical portion, the squama frontalis, corresponding to the region known as the forehead.
Mandible: The mandible forms the lower jaw. The mental protuberance is the bony protuberance of the jaw that forms the chin.
Maxilla: The maxilla forms the upper jaw and is located above the mandible and below the orbits. The frontal process of the maxilla projects upwards by the side of the nose, and forms part of its lateral boundary.
Nasal bones: The nasal bones are two small oblong bones, varying in size and form in different individuals; they are placed side by side at the middle and upper part of the face, and form, by their junction, the “bridge” of the nose.
Nasion: The intersection of the frontal bone and the two nasal bones, a depressed area directly between the eyes and superior to the bridge of the nose.
Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.
Orbit: The bony cavity in the skull to contain the eyeball.
Parietal bones: The parietal bones are the bones that, when joined together, form the roof and sides of the cranium.
Temporal bones: The temporal bones are situated on the bases and sides of the skull, and support that part of the face known as the temple.
Zygomatic bones: The face includes two zygomatic bones, located in the upper and lateral parts of the face and forming the prominence of the cheek.

### 4.7.3.3 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.
Larynx: The larynx, or voice box houses the vocal folds and connects the inferior part of the pharynx (hypopharynx) with the trachea.
Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.
Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.
Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.7.4 Patient Interface

Anti-asphyxia valve (AAV): The component or sub-assembly of a mask system that, by opening to atmosphere in a failsafe manner, reduces the risk of excessive CO2 rebreathing by a patient.
Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, c.g. about 360 degrees. In certain forms an elbow may be removable from a mating component, e.g. via a snap connection. In certain forms, an elbow may be assembled to a mating component via a one-time snap during manufacture, but not removable by a patient.
Frame: Frame will be taken to mean a mask structure that bears the load of tension between two or more points of connection with a headgear. A mask frame may be a non-airtight load bearing structure in the mask. However, some forms of mask frame may also be air-tight.
Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.
Membrane: Membrane will be taken to mean a typically thin clement that has, preferably, substantially no resistance to bending, but has resistance to being stretched.
Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.
Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ clement per se.
Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.
Stiffener: A stiffener will be taken to mean a structural component designed to increase the bending resistance of another component in at least one direction.
Strut: A strut will be taken to be a structural component designed to increase the compression resistance of another component in at least one direction.
Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.
Tie (noun): A structure designed to resist tension.
Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 4.7.5 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 4.7.5.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 4.7.5.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign.

Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical-topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 4.7.5.3 Space curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the Tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 3O and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 4.7.5.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 4.8 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A positioning and stabilising structure for a patient interface configured to deliver breathable gas at a therapeutic pressure of at least 4 cmH₂O above ambient air pressure to an entrance to a patient's airways, the positioning and stabilising structure comprising:
a rear portion configured to overlie a posterior region of the patient's head;
first and second side straps as claimed in claim 83,
wherein each of the first and second side straps is configured, in use, to extend across a respective cheek region of the patient's head, and each having a posterior end connected to a respective side of the rear portion and an anterior end configured to connect directly or indirectly to a seal-forming structure of the patient interface, the seal-forming structure being configured to form a seal with a region of the patient's face surrounding an entrance to the patient's airways.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20.-32. (canceled)

33. A patient interface comprising:
a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a vent to allow a continuous flow of gases exhaled by the patient from an interior of the plenum chamber to ambient, said vent being sized and shaped to maintain the therapeutic pressure in the plenum chamber in use; and
a positioning and stabilising structure as claimed in claim 1.

34.-82. (canceled)

83. A side strap for a positioning and stabilising structure for a patient interface configured to deliver breathable gas at a therapeutic pressure of at least 4 cmH2O above ambient air pressure to an entrance to a patient's airways, the side strap comprising a rigidiser arm positioned inside a sleeve, and wherein the rigidiser arm and the sleeve are connected together so that substantially no part of the sleeve can move relative to the rigidiser arm.

84. A side strap as claimed in claim 83, wherein the side strap is tapered such that the posterior end is wider than the anterior end.

85. A side strap as claimed in claim 83, wherein the sleeve is formed so as to be flexible.

86. A side strap as claimed in any claim 83, wherein the sleeve is formed from a textile material.

87. A side strap as claimed in claim 83, wherein the sleeve comprises an outer sleeve and an inner sleeve, the inner sleeve positioned inside the outer sleeve and the rigidiser arm positioned inside the inner sleeve.

88. A side strap as claimed in claim 87, wherein the inner sleeve is thicker than the outer sleeve.

89. A side strap as claimed in claim 87, wherein the inner sleeve is denser than the outer sleeve.

90. A side strap as claimed in claim 87, wherein the inner sleeve is formed with a denser weave or knit pattern than the outer sleeve.

91. A side strap as claimed in claims 83, wherein the side strap comprises one or more cushion portions positioned inside the sleeve.

92. A side strap as claimed in claim 91, wherein the side strap comprises two cushion portions positioned inside the sleeve on opposite lateral sides of the rigidiser arm.

93. A side strap as claimed in claim 83, wherein the rigidiser arm is formed from a heat-activated material, and wherein the heat-activated material is bonded with the sleeve.

94. A side strap as claimed in claim 83, wherein the rigidiser arm is constructed in a shape that makes deformation of the rigidiser arm in one or more directions difficult, but the rigidiser arm is flexible in at least one direction.

95. A side strap as claimed in claim 83, wherein the rigidiser arm has a length that is significantly greater than its width, and a width that is significantly greater than its height.

96. A side strap as claimed in claim 83, wherein the rigidiser arm is formed with a curved shape to substantially correspond to the shape of respective sides of the patient's head.

97. A side strap as claimed in claims 83, wherein the rigidiser arm is curved when viewed from a direction perpendicular to one of the surfaces of the rigidiser arm facing towards or away from the patient's face in use.

98. A side strap as claimed in claim 97, wherein the rigidiser arm curves upwardly so that the superior edge of the rigidiser arm is concave and the inferior edge of the rigidiser arm is convex.
